(19) United States
(12) Patent Application Publication
(10) Pub. No.: US 2009/0298186 A1
(43) Pub. Date: Dec. 3, 2009

(54) Method to Assess Stability of Proteins

(76) Inventors: Michael Brigham-Burke, King of Prussia, PA (US); Eilyn R. Lacy, Radnor, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/394,714

(22) Filed: Feb. 27, 2009

Related U.S. Application Data
(60) Provisional application No. 61/032,633, filed on Feb. 29, 2008.

(51) Int. Cl. G01N 33/68

(52) U.S. Cl. 436/86

(57) ABSTRACT

A method for determining conformational stability of proteins detects the change in free sulfhydryls accessible to reaction with a fluorescent probe after combined chemical and thermal denaturation. The method is useful in any application where the stability and integrity of a protein preparation is useful information. The method can be used to screen protein variants for desirable stability profile.

### CROSS REFERENCE TO RELATED APPLICATION

This application claims the benefit of U.S. Provisional Application Ser. No. 61/032,633, filed 29 Feb. 2008, the entire contents of which is incorporated herein by reference in its entirety.

### BACKGROUND OF THE INVENTION

1. Field of the Invention

The invention relates to methods of evaluating the structural stability of proteins and peptides to enable characterization of the proteins and peptides and, more specifically, a method of evaluating the structural stability of protein preparations by measuring the free sulfhydryl content.

2. Description of the Related Art

Proteins are characterized by primary structure, e.g., the linear sequence of amino acid residues of the polypeptide chain(s); secondary structure, folds and twists (beta-pleated sheets and alpha-helical coils) adopted by the polypeptide chain; tertiary structure which is the overall 3-dimensional arrangement of the polypeptide chain; and, in some cases, the quartenary structure, which is the manner in which multiple polypeptides associate to form a functional complex. Protein conformation is stabilized by intramolecular electrostatic, hydrophobic interactions and, in some cases, disulfide bonds. Among the intra- and inter-chain stabilizing forces, disulfide bonds represent the only covalent linkage and are the strongest of the three. Therefore, the presence or absence of disulfide bonds between the side-groups of cysteine residues is of critical importance to the correct folding and stabilization of proteins and maintenance of their intended bioactivity. Although a protein or peptide may comprise the correct primary and even secondary structural elements, it will not be chemically or structurally stable unless it has formed the correct network of disulfides.

Functional protein products, such as industrial enzymes and biologic therapeutics, require enhanced structural stability. Further, whether the protein is produced by chemical synthesis or by recombinant expression, stability must be maintained throughout purification, formulation, and shelf-life.

There is a need in the art of protein engineering and biopharmaceutical manufacturing for methods to assess protein stability.

### SUMMARY OF THE INVENTION

The present invention comprises a method to assess protein stability in response to a denaturing condition using a change in free SH in the protein preparation using, for example, fluorescent detection. In one embodiment of the method of the invention the detection reagent is maleimide capable of forming a fluorescent thioester upon reaction with a protein —SH and the denaturing condition is heat and a chemical denaturant, such as guanidinium hydrochloride.

The method of the invention is applicable to analysis of any functional protein comprising at least two cysteine residues and at least a disulfide bond. The method can be used to assess stability of complex proteins or protein mixtures. In one embodiment of the invention, the method can be applied to assessing the stability of polyclonal antibody preparations, monoclonal antibodies, antibody fragments, such as Fabs, antibody derived constructs, such as scFv and single antibody domains, protein therapeutics which may be enzymes, industrial enzymes, peptides, and protein digests and any variant or derivative thereof, provided that these compositions contain cysteine residues capable of forming a disulfide bond. In another aspect of the invention, the method uses sample volumes and minimized protein consumption in a high throughput format for screening and selection among multiple protein variants.

The method of the invention may be applied to any aspect of protein product research or development where information on protein structural stability is a useful parameter. In various aspects of the invention, the method is used to determine intrinsic stability during screening of protein variants or alternate candidates produced in early stages of the selection process, determine intrinsic stability of candidates in the final selection process, determine sample stability under different formulations in pharmaceutical development, or determine sample stability under different storage conditions.

### BRIEF DESCRIPTION OF THE SEVERAL VIEWS OF THE DRAWING

FIG. 1 shows the principle disulfide bonds in IgG1 and IgG4 class/subclass antibodies that stabilize the intrachain and interchain domain structures.

FIG. 2 shows typical calibration curves for free sulfhydryls: BSA (circles) and N-acetyl-L-cysteine (triangles) under nondenaturing conditions at 25° C. (closed symbols) and denaturing conditions at 50° C. (open symbols).

FIGS. 3A-D show the spectra of various —SH containing preparations used as standards: FL signal for (A) N-acetyl-L-cysteine under nondenaturing conditions, (B) N-acetyl-L-cysteine under denaturing conditions, (C) BSA under nondenaturing condition, and (D) BSA under nondenaturing conditions after reaction with NPM at concentrations from 0.02 to 17 uM or 25 uM (bottom to top curves).

FIGS. 4A and 4B show the FL signal (cps) for amino acids in the presence of NPM or for protein solutions containing all reagents but NPM. All the samples were prepared at 3.5 uM in PBS and the reactions were performed at 25° C. In panel (A), triangles and circles correspond to N-acetyl-L-cysteine and phenylalanine after reaction with NPM, respectively. The curves for the reaction with glycine, the control with buffer and the control for N-acetyl-L-cysteine in the absence of NPM are represented by positive sign, lines and squares, respectively. In panel (B), circles correspond to BSA after reaction with NPM, while the squares and X correspond to BSA in the absence of NPM and the buffer control (all reagents except protein).

FIG. 5 is a graph wherein the correlation between free sulfhydryls (—SH) and structural stability (structural stability determined by CD thermal denaturation experiments) is determined: the —SH content of IgGs versus thermal stability expressed as the Tm for the first temperature dependent structural transition. The Tm was determined in PBS by monitoring the CD signal at 216 nm while increasing the temperature from 60 to 95° C. The content of free sulfhydryls was obtained under nondenaturing (closed squares) and denaturing (open squares) conditions.

FIG. 6 shows the correlation between free sulfhydryls (—SH) and structural stability (structural stability determined by DSC experiments): the —SH content of IgGs versus thermal stability expressed as the Tm for the first temperature dependent structural transition as determined in PBS by DSC obtained under nondenaturing (closed squares) and denaturing (open squares) conditions with incubation of the reaction mixture at 37° C.

### DETAILED DESCRIPTION OF THE INVENTION

### Abbreviations

Abs antibodies, polyclonal or monoclonal; CD circular dichroism; DMF dimethyl formamide; DSC differential scanning calorimetry; Gdn-HCl guanidinium hydrochloride; Fab antibody fragment comprising binding domain contributed by both heavy and light chain domains; Ig immunoglobulin; Mab monoclonal antibody; FL fluorescence or fluorescence units (arbitrary); MBB monobromobimane; NPM N-pyrenyl maleimide; scFv single chain fragment comprising antibody variable regions which is an engineered fusion protein comprising a single polypeptide chain;

### DEFINITIONS

A “sulfhydryl” “sulfhydryl group,” “free sulfhydryl,” “thiol group,” “—SH,” or “free —SH,” as used herein refers to the chemical moiety comprising a sulfur atom linked to a carbon atom and also bonded to a single hydrogen. A “protein” shall mean a peptide or polypeptide molecule that may comprise a single subunit or multiple subunits.

By “chemical denaturant” is meant an agent known to disrupt non-covalent bonds and covalent interactions within a protein, including hydrogen bonds, electrostatic bonds, Van der Waals forces, hydrophobic interactions, or disulfide bonds. Chemical denaturants include guanidinium hydrochloride, guanadinium thiocyanate, urea, acetone, organic solvents (DMF, benzene, acetonitrile), salts (ammonium sulfate lithium bromide, lithium chloride, sodium bromide, calcium chloride, sodium chloride); non-ionic and ionic detergents, acids (e.g. hydrochloric acid (HCl), acetic acid (CH₃COOH), halogenated acetic acids), hydrophobic molecules (e.g. phosopholipids), and targeted denaturants (Jain R. K and Hamilton A. D., Angew. Chem. 114(4), 2002).

By “denature” or “denaturation” of a protein is meant the process where some or all of the three-dimensional conformation imparting the functional properties of the protein has been lost with an attendant loss of activity and/or solubility. Forces disrupted during denaturation include intramolecular bonds, including but not limited to electrostatic, hydrophobic, Van der Waals forces, hydrogen bonds, and disulfides. Protein denaturation can be caused by forces applied to the protein or a solution comprising the protein such as mechanical force (for example, compressive or shear-force), thermal, osmotic stress, change in pH, electrical or magnetic fields, ionizing radiation, ultraviolet radiation and dehydration, and by chemical denaturants.

### Description Overview

Measurement of protein stability and protein lability can be viewed as the same or different aspects of protein integrity. Proteins are sensitive or “labile” to denaturation caused by heat, by ultraviolet or ionizing radiation, or changes in the ambient osmolarity and pH if in liquid solution. Thermal denaturation methods commonly used in protein studies provide information about the structural stability of the molecules but do not provide information about their chemical stability. While stability testing for clinical supplies typically uses only heat (elevated temperatures over periods of months to years), the method of the invention requires a shortened period of testing of as little as minutes to hours. In addition, thermal stability testing requires typically more than 300 ug of protein, which limit their use in high throughput applications (HTP). Structural stability can also be assessed using more sophisticated techniques, such as circular dichroism and differential scanning calorimetry.

A symptom of an unstable protein is the loss of disulfide bonds (Proba et al., J. Mol. Bol. 265:161-172, 1997, Kikuchi et al., Biochemistry 25:2009-2013, 1986). Disulfide bonds are labile at neutral pH, forming two free sulfhydryls (—SH). Sulfhydryl reduction or exchange is limited in stabilized proteins because the sulfhydryls are held in close proximity to each other by electrostatic or hydrophobic interactions or are buried within the protein structure and are not exposed to the external reactive environment (Magnusson et al., Molecular immunology 10: 709-717, 1997). Free sulfhydryls may result when cellular processes do not correctly fold the protein or be due to modifications in the primary sequence that result in disruption of the disulfide bond (Zhang and Czupryn, Biotechnology Progress 18: 509-513, 2002). Thus, the presence of free sulfhydryls implies the loss of an element necessary to maintain the tertiary and/or quaternary structures of proteins and therefore can be taken as an overall indicator of reduced structural stability of the protein.

It has been demonstrated in the present invention, using free —SH detection, that the relationship between fractional —SH and protein stability can be used to assess overall conformational integrity of a protein. Measurement of —SH has heretofore not been attempted as a routine method of assessing protein stability, possibly because the inherent number of cysteine residues in a protein is statistically small and the fraction of the product that is misfolded may be minimal, or the fact that —SHs may form only transiently (Chang et al., Anal Biochem 342:78-75, 2005; Zhang and Czupryn, Biotechnol Prog 18: 509-513, 2002). For example, Zhang and Czupryn report that in a purified monoclonal antibody under native conditions 0.02 mole of sulfhydryls per mole of antibody (0.06% of the total 32 cysteines) are transiently present. Under denaturing conditions, as much as 0.1 mole of sulfhydryls per mole antibody (0.3%) are present. Thus, a method that relies on the change in —SH in a relatively stable protein such as an antibody must be sensitive enough to provide for a low level of sulfhydryls.

Ellman's test (Ellman, Archives of Biochemistry and Biophysics 82:70-77, 1959) uses 5,5′-dithiobis(2-nitrobenzoic acid) or DTNB, which upon reaction with a free sulfhydryl, has an extinction coefficient of 13,600 M⁻¹ cm⁻¹ at 412 nm. Another reagent reactive with free —SH is bromobimane (CAS Number: 71418-44-5, monobromobimane, mBBr) (Kosower and Kosower. 143:76-84, 1987) which can be used to titrate cysteinyl residues in proteins, but has proven to produce high background responses that interfere with the free sulfhydryl analysis (Wright and Viola, Analytical Biochemistry 265: 8-14, 1998). New research has shown that maleimide derivatives, which produce fluorescent products upon reaction with free sulfhydryl, may be a better alternative for the analysis of free sulfhydryl in proteins (Winters et al., Analytical Biochemistry 227: 14-21, 1995; Zhang and Czupryn, 2002 supra).

Accessible free sulfhydryls (—SH) in proteins and peptides react with maleimides to form a thioester through the formation of a C—S covalent bond. N-(1-pyrenyl)maleimide (NPM) is one example of a maleimide, which when free in solution, has essentially no fluorescence, but becomes fluorescent upon formation of the thioester (Woodward et al., Journal of Biochemical and Biophysical Methods 26: 121-129, 1993); proteins (Winters et al., Analytical Biochemistry 227: 14-21, 1995). The present invention uses fluorescent thioester forming maleimides in a method that can be used to investigate the chemical stability of proteins and peptides, expressed in terms of free sulfhydryl (—SH) content.

For example, human and bovine serum albumin (HSA and BSA) contain 35 cysteines of which are 17 disulfide bonds and one —SH (Curry et al., Nature Structural Biology 5:827-835, 1998; He and Carter, D. C. Nature, 358, 209-215, 1992) and BSA (Ferrer et al., Biophys J. 80:2422-2430, 2001). In the development of the present method, native BSA was used as a suitable standard for quantification of free sulfhydryl in biomolecules. Using BSA, a correlation between the —SH and the structural integrity of the protein being studied was established. To further correlate these results with knowledge of the structural stability of the protein, circular dichroism (CD) or differential scanning calorimetry (DSC) was performed under conditions of thermal denaturation.

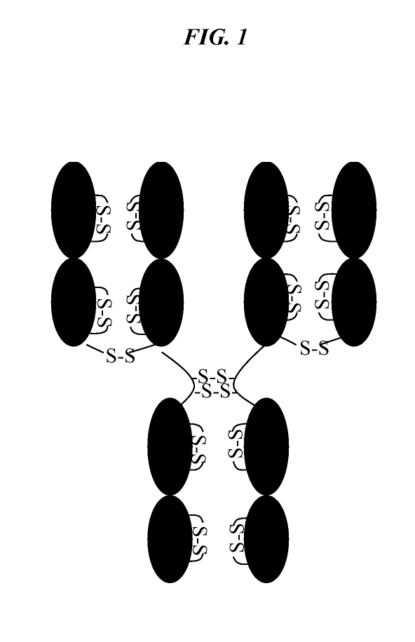

In another embodiment of the invention, a set of IgGs (antibodies) were used to demonstrate the differences in chemical and thermal stability of structurally related protein species or variants. An IgG is a homodimer of a heterodimer consisting of a heavy chain and a light chain. Each heterodimer comprises a heavy chain with four immunoglobulin domains and light chain with two immunoglobulin domains where each domain is stabilized by a disulfide bond. The heavy chain and light chain are cross-linked by a disulfide bond and each heterodimer is further crosslinked by at least one and sometimes three or more disulfide bonds (FIG. 1). In the IgG1 and IgG4, the most commonly produced antibodies; the heavy chains are held together by two disulfide bonds. Therefore, there are at least 16 disulfide bonds or 32 potential sulfhydryls per IgG1 or IgG4 molecule. Although all cysteines should be engaged in a disulfide bridge in a properly folded antibody, free —SH have been found in samples of monoclonal antibodies (Zhang and Czupryn, 2002, supra). Free —SH in antibodies implicate the presence of free light and heavy chains which compromises the functional integrity of the antibody.

The method of the present invention can thus be used advantageously to provide information about the chemical stability of the disulfide bond pattern of a subject protein or the method can be used empirically to rank and select among a series of variants or varied preparations on the basis of their overall stability. In addition, the present method uses a significantly low amount of protein (as low as 50 ug) making it amenable to small scale and high-throughput screening applications.

The invention is based on the discovery of conditions under which the change in —SH in a protein preparation can be made sensitive enough to enable practicing the method with small quantities of protein for determination of —SH possible. The increased sensitivity is due, in part, to the use of a fluorescent probe, such as MBB or NPM, whose fluorescence is significantly enhanced upon reaction with free sulfhydryls (—SH) forming a thioester. Thus, the use of a fluorescence-based detection probe is more sensitive than the colorimetric chromophore of DTNB (Ellman's reagent) which is 1.36×10⁴ M⁻¹ cm⁻¹ at the A maximum of 412 nm. As A=ebc, where e is the extinction coefficient of the absorbing species, b is the pathlength, and c is the Molar concentration in the solution being measured, a sample containing 150 ug of an antibody in 1 ml is approximately a 1 uM solution and, if 1-SH was present, would give an Absorbance reading of 0.0136.

In fluorescence spectroscopy, the fluorescent radiation (F) can be expressed as F=2.3K′ ebc Po (Skoog et al., Fundamentals of Analytical chemistry, page 605, 1996), which can be reduced to F=K″ ebc. For a particular molecule at a specific wavelength e is constant; therefore, it can be expressed F=Kbc.

Using the fluorescence signal at 376 nm for NPM upon reaction with BSA, we calculated that the constant (K) is 6×10¹²; therefore, F=6×10¹²*bc. As compared to A=1.36×10⁴*bc, this shows that the signal for NPM is significantly more sensitive than DTNB. In addition, in contrast to methods using other fluorescent reagents such as the bromobimane, the method described here does not produce high background and does not require a purification step after the reaction when using NPM as a fluorophore.

In addition, the fluorescence signal at 376 nm is about two-fold greater than at 380 nm under the conditions employed in the embodiment above. The working volume can be scaled to only 300 mL, which makes the assay compatible for automation in 96-well microtiter plates. The method is adaptable to a range of pH conditions (about pH 5 to about pH 8.5) and denaturants and will not be affected by amine containing buffer constituents or sugars typically used in formulations.

Further, the sensitivity of the present method allows the use of mildly denaturating conditions to be applied to the subject protein preparation, such that complete denaturation is unnecessary. In the examples described herein, as little as 1 mole of —SH per mole of protein may be detected, which may represent the structure of a single domain in a complex protein (e.g., an antibody). In one embodiment, guanidinium salts have been used as a chemical denaturant in combination with mild thermal stress to cause changes in protein conformation detectable by measurement of —SH.

In other aspects of the invention, other denaturing substances or energy sources can be applied to the sample to impose a denaturing force on the sample, which may include mechanical shear force imposed by small pore-size filtration, ultraviolet radiation, ionizing radiation, such as by gamma irradiation, chemical or heat dehydration, or any other action or force that may cause protein denaturation.

### Use of the Method

The method of determining protein conformation stability and integrity disclosed herein is particularly useful in industrial settings where quantities of active proteins are desired to be produced. Due to the requirement for small sample amounts and rapid processing times, in one aspect of the invention, the method of determining protein stability can be used as a method to select among therapeutic protein candidates made in small amounts prior to scale up efforts.

The method of the present invention may also be used as an additional method to discriminate between proteins with other similar properties, such as Tm, but which denature at different rates. By discriminating between proteins on the basis of their kinetics of unfolding or denaturation, that is the rate at which the protein reacts with NPM, an alternate parameter for measuring protein stability is achieved. The difference in reaction rates can be measured using either manual or automated methods described above and recording signal strength over time. Kinetics can be analyzed using standard curve fitting algorithms and e.g. the time at which the rate of unfolding is maximal, and these parameters can be compared or ranked to complete the determination of absolute or relative stability as the situation warrants.

While having described the invention in general terms, the embodiments of the invention will be further disclosed in the following examples.

### Example 1

### Preparation of Standards

Calibration curves for different concentrations of —SH were prepared using either N-acetyl-L-cysteine or BSA as a standard from 0.02 to 17 μM under denaturing and nondenaturing conditions. The stock solutions for the standards for the calibration curves were prepared in Dulbecco's phosphate-buffered saline (D-PBS), pH 7.3. Buffers were deoxygenated and degassed by sonication under vacuum and then bubbled with argon. NPM was prepared at 10 μM concentration in dimethylformamide (DMF).

For the reactions, 50 μL of the appropriate BSA (or N-acetyl-L-cysteine) solution were mixed with 250 μL of D-PBS, pH 7.3 with or without 6M Gdn-HCl to the desired final concentration. Three μL of 10 μM NPM were added to this mixture, and the samples were incubated either 5 min, 1 h or 2 h at RT or 25° C. for solutions in D-PBS; and incubated at RT, 37° C., 40° C., 50° C. or 60° C. for those with Gdn-HCl. NPM was added slowly to avoid cloudiness in D-PBS. The reaction was stopped by adding 50% acetic acid to a concentration of 0.8%.

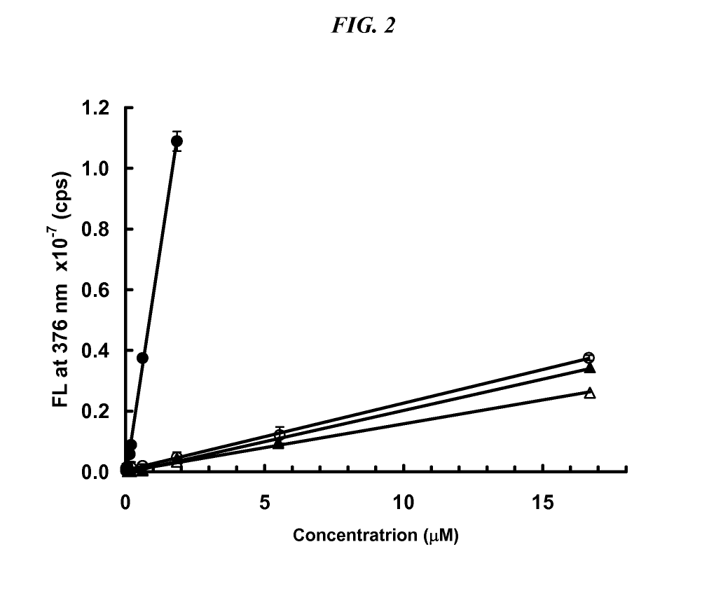

It was found that the signal was optimal for nondenatured samples using 2 h at 25° C. in D-PBS and for denatured samples, incubation for 2 h at 37° C. with Gdn-HCl. The fluorescence emission spectrum of the mixture was then obtained using a Fluoromax-3 fluorometer and 3 μm path-length fluorometer cells. The samples were excited at 330 nm and the spectra were collected from 350-450 for the initial experiments, using 4 nm excitation and emission slits. Once the wavelength of maximal sensitivity was established the data collection was reduced to 370-380 nm to reduce the data collection time. The calibration curves were prepared by plotting the fluorescence signal at 376 nm versus micromolar concentration of BSA or N-acetyl-L-cysteine. The calibration curves used were in the linear range of the curve which was from 0.03 uM to 1.7 uM for BSA under nondenaturing conditions, and up to 17 uM for the others (FIG. 2).

The calibration curves of N-acetyl-L-cysteine were used to confirm the content of —SH per mole of BSA and to determine the effect of the presence of various proteins on the intensity of the signal observed. Using the slope for N-acetyl cysteine in 5M Gdn-HCl, it was determined that under denaturing conditions there is one mole —SH/mol of BSA. This is in agreement with the crystal structure and sequence of serum albumin (Curry, et al., Nature Struc Biol 5:827-835, 1998; He and Carter, D. C. Nature 358: 209-215, 1992), and validates the use of BSA as a standard for quantitation of free sulfhydryl in biomolecules.

The difference in the slope for the calibration curve with BSA under nondenaturing conditions, 6.5×10⁶ fluorescent units (cps)/uM, whereas under denaturing conditions was 3.8×10⁵ fu/uM. This may be due to quenching of the reacted NPM signal once it is exposed to solvent under denaturing conditions in comparison to the signal within the protein environment in nondenaturing conditions. For N-acetyl-L-cysteine the signals are similar under both conditions because the —SH are always completely exposed to solvent. Owing to these differences, BSA represents a more appropriate standard for quantitation of free sulfhydryl in biomolecules.

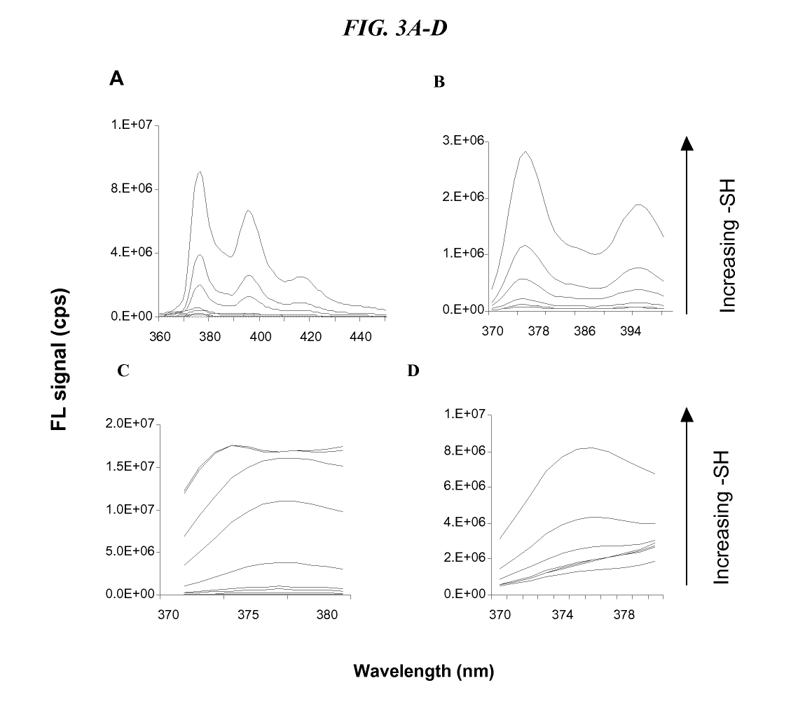

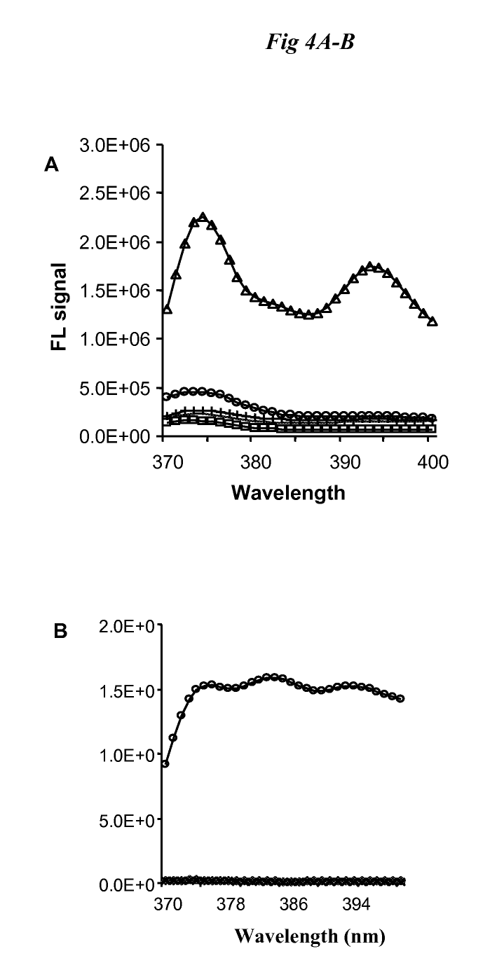

Control experiments were performed for other conditions to confirm that the signal observed was a result of —SH reaction with NPM and not an acquired signal from NPM when in contact with hydrophobic patches in the protein surface. FIGS. 3A-D show the spectra of various —SH containing standards: FL signal for (a) N-acetyl-L-cysteine under nondenaturing conditions, (b) N-acetyl-L-cysteine under denaturing conditions, (c) BSA under nondenaturing condition, and (d) BSA nondenaturing conditions after reaction with NPM at concentrations from 0.02 to 17 uM or 25 uM (bottom to top curves). FIG. 4 shows the FL signal (cps) for amino acids in the presence of NPM or for protein solutions containing all reagents but NPM. All the samples were prepared at 3.5 uM in PBS and the reactions were performed at 25° C. FIGS. 4A and 4B show that a fluorescent signal from NPM is observed only in the presence of —SH and no significant signal is observed for the mixtures of NPM with phenylalanine, glycine, buffer alone or for protein in the absence of NPM.

### Example 2

### Assay Conditions

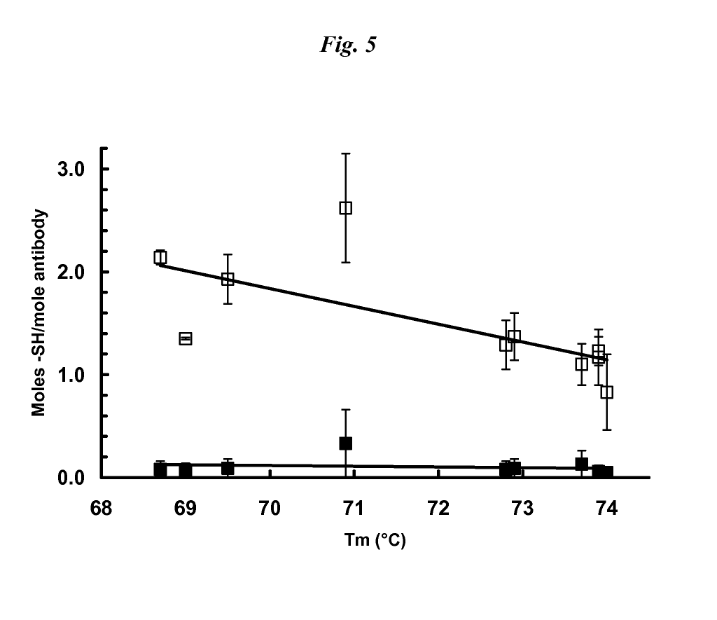

To determine the optimal conditions for determination of —SH under denaturing conditions, several proteins, BSA and three antibodies were subjected to various amounts of thermal denaturation. Adalimumab is a human anti-TNF antibody, infliximab is a murine-human chimeric anti-TNF antibody, and MAB6 is a human engineered anti-cytokine antibody. The signal obtained for standards and samples analyzed at 60° C., 50° C., 40° C. and 37° C. were compared. The temperature at which the net signal (sample signal minus buffer) was maximal was obtained is 37° C. (FIG. 5). In addition, the effect of reaction time was tested at 5 min, 1 h and 2 h. The optimal reaction time, where signal strength reached a plateau, was 2 h. Experiments at pH 6.0 were also performed. Although signal was obtained, it was significantly reduced as compared to pH 7.3.

The best results were produced when samples were prepared in a non-reducing buffer (such as Dulbecco's phosphate-buffered saline (D-PBS), pH 7.3) containing 3 mM EDTA; the buffer was degassed and, optimally, also saturated with argon; NPM was prepared at 10 mM concentration in dimethylformamide (DMF) using silicon and latex free syringes. For the reactions, 50 uL of the appropriate sample was mixed with 250 uL of D-PBS, pH 7.3 containing 3 mM EDTA or 250 μL of D-PBS, pH 7.3 containing 3 mM EDTA and containing 6 M Gdn-HCl. These sample preparations were incubated at 25° C. (non-denaturing exp) or 37° C. (for denaturing exp) for 1 h. After this incubation, 3 μL of 10 μM NPM was added to each solution. The reaction mixtures were incubated for 2 h at 25° C. (non-denaturing exp) or 37° C. (for denaturing exp). The reaction was stopped with 5 uL of 50% acetic acid. The fluorescence emission is obtained at 330 nm excitation and 376 nm emission using 4 nm excitation and emission slits. BSA standards (0.02 to 17 μM) were prepared and used as reference values using the same procedures used for the samples. The calibration curve generated using the BSA standards under each separate condition (nondenaturing and denaturing) are used to determine the —SH content of the test samples for the respective condition. The proteins are ranked for stability on the basis of the —SH content under denaturing conditions.

The temperature dependence of the FL at 376 nm for —SH reacted NPM under denaturing conditions data are given in Table 1. PBS and PBSn correspond to two control samples (no protein added) prepared with two different lots of NPM. Adalimumab 1-3 correspond to three different lots of adalimumab (prepared from prepackaged samples of HUMIRA™, Abbott Pharmaceuticals, Abbott Park, Ill.). The samples were analyzed at 3.5 μM in PBS containing 5M Gdn-HCl and 3 mM EDTA and the values multiplied by 10⁻⁶ fu per sample

TABLE 1

| Sample name | 60° C. | 50° C. | 37° C. |
|---|---|---|---|
| PBS | 1.50 ± 0.14 | 1.40 ± 0.10 | 1.50 ± 0.05 |
| PBSn | 1.78 ± 0.04 | 1.44 ± 0.17 | 1.49 ± 0.03 |
| BSA | 3.02 ± 0.21 | 2.77 ± 0.16 | 3.91 ± 0.09 |
| adalimumab 1 | 2.91 ± 0.10 | 2.50 ± 0.11 | 3.37 ± 0.10 |
| adalimumab 2 | 2.80 ± 0.14 | 2.68 ± 0.06 | 3.52 ± 0.10 |
| adalimumab 3 | 2.97 ± 0.01 | 2.44 ± 0.14 | 3.40 ± 0.10 |
| infliximab | 2.78 ± 0.05 | 2.16 ± 0.15 | 2.87 ± 0.05 |
| MAB6 | 2.79 ± 0.05 | 2.33 ± 0.03 | 2.96 ± 0.12 |

### Example 3

### Stability of Structurally Related Proteins

Human serum IgG1, and IgG4 antibodies (lambda and kappa light chains) were obtained from Sigma. Monoclonal antibodies; CDG1, a humanized murine Mab which has a human IgG4 heavy chain and kappa LC constant regions (IgG4,κ); Mab13 is a human IgG1 with lambda light chain, Mab59, Mab12 and Mab9.5 are human IgG1 with kappa light chains; and Mab41 and Mab48 are humanized murine Mabs with IgG4 heavy and kappa light chains. All of the Mabs had unique binding specificity and unique hypervariable domains (CDR) domains.

For analysis of free sulfhydryl, antibodies were prepared at 1 μg/mL (6.7 uM) in D-PBS, pH 7.3 or Tris buffer, pH 7.2 and diluted to 1.1 uM using the appropriate reaction buffer. Solutions at 3 μg/mL were also used, but the data show that the results obtained are comparable to those obtained at 1 μg/ml (using IgG1 lambda and kappa as controls). 50 uL of these solutions of protein were treated following the same procedure used for the reaction of BSA with NPM (previous two sections) for nondenaturing and denaturing conditions. Similar to the standards, maximum emission and sensitivity to free sulfhydryl content was obtained at 376 nm. The micromolar concentration of free sulfhydryl in the antibodies was determined using the calibration curves obtained for BSA. The fraction of —SH per mol of protein was determined on the basis of the final concentration of the sample in the reaction mixture.

To investigate the structural stability of the antibodies studied, the samples were prepared at 1 or 3 μM in D-PBS and their circular dichroism (CD) spectra were recorded from 195 to 260 nm. Thermal denaturation experiments were performed by recording the CD signal at 216 nm while increasing the temperature at 1° C./min. The Tm were obtained from the maxima of the first derivative of the melting profiles. Thermal denaturation experiments were also performed using differential scanning calorimetry DSC.

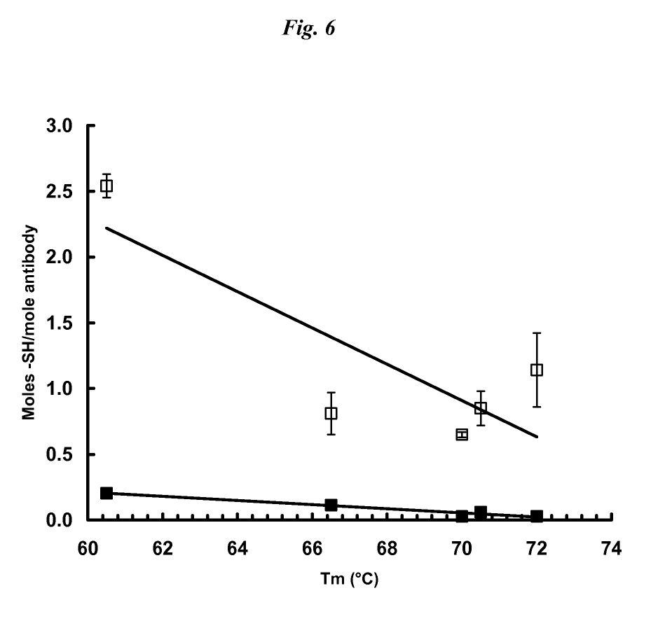

FIGS. 5 and 6 show a plot of moles of —SH per mAb versus the temperature for the first temperature dependent structural transition of the antibodies. These show the relationship between —SH content and the structural stability of antibodies. Tables 2, 3 and 4 show details of the results obtained. Full access of NPM to —SH buried in the interface of different domains or in the interior of folded proteins is achieved under denaturing conditions (5M Gdn-HCl and temperature). The present method of —SH analysis of polyclonal IgG1 antibodies revealed that serum IgG1 lambda is less chemically stable than IgG1 kappa as demonstrated by a significantly larger content of —SH in IgG1 lambda than in IgG1 kappa (Tables 2, 3 and 4). For these molecules, chemical stability correlates with thermal stability as the molecule with the larger content of —SH has the lower Tm.

Similarly, —SH content in Mab13 is larger than free sulfhydryl content on Mab9.5 (Tables 2, 3). These results were corroborated by SEC. Under denaturing conditions, a larger amount of antibody fragments (light chain, heavy chain and half mAb) is detected for MAB13 as compared with MAB9.5. To perform SEC analysis the antibodies were prepared at 1 μg/mL in D-PBS, 3 M Gdn-HCl or in 3M guanidinium thiocyante, pH 7.2. The samples in Gdn-HCl or guanidinium thiocyante were incubated at 40° C. for at least 15 min prior to injection and separation using a Superdex 75 gel filtration column (Amersham Biosciences). Thermal denaturation analysis (Table 2) shows that the first structural transition due to temperature denaturation of MAB13 occurs 5° C. lower than for MAB9.5.

Another study case involved the study of IgG4 antibodies. Under nondenaturing conditions CDG1, Mab41 and Mab48 show similar chemical stability (Table 2); however, under denaturing conditions the free sulfhydryl in Mab41 and Mab48 are more accessible for reaction with NPM. The inherent instability of these two antibodies relative to CDG1 is confirmed by the Tₘ obtained by CD thermal analysis.

The correlation between chemical and structural stability (shown in FIGS. 5 and 6, Tables 1-3) demonstrates that chemical stability, in the form of moles —SH/mole of protein formed under denaturing conditions, can be used to infer or predict the structural stability of proteins. Therefore, proteins or peptides which are structural analogs can be ranked as to predicted stability on the basis of their —SH content using the present assay.

Table 2 shows the molar fraction of —SH in antibodies under denaturing conditions (Dulbecco's phosphate-buffered saline, pH 7.3 containing 5 M Gdn-HCl) calculated using a calibration curve generated using N-acetyl cysteine as a standard. The samples were incubated for 1 h at 40° C.

TABLE 2

| | Free sulfhydryl |
|---|---|
| Antibody | (mol —SH/mol Mab) |
| IgG1 kappa | 0.15 |
| IgG1 lambda | 3.20 |
| MAB9.5 | 0.02 |
| MAB13 | 0.46 |

Table 3 show the molar fraction of —SH in antibodies under non-denaturing conditions (Dulbecco's phosphate-buffered saline, pH 7.3) and denaturing conditions (Dulbecco's phosphate-buffered saline, pH 7.3 containing 5 M Gdn-HCl) calculated using a calibration curve generated using BSA as a standard. The reaction mixtures for this set of samples were incubated for 2 h at 50° C. Table 3 also shows the melting temperatures (Tm) of the first temperature dependent structural transition of these antibodies as determined by CD.

TABLE 3

| | Tm, first | | |
|---|---|---|---|
| | thermal | Free SH | Free SH |
| | transition | Non-denaturing | Denaturing |
| Antibody | (° C.) | (mol —SH/mol Mab) | (mol SH/mol Mab) |
| IgG1 kappa | 73.9 | 0.0481 ± 0.0019 | 1.23 ± 0.14 |
| IgG1 lambda | 70.9 | 0.333 ± 0.010 | 2.62 ± 0.53 |
| Mab12 | 74.0 | 0.0513 ± 0.0038 | 0.827 ± 0.368 |
| MAB9.5 | 73.9 | 0.0592 ± 0.0034 | 1.17 ± 0.27 |
| MAB13 | 69.0 | 0.0648 ± 0.0028 | 1.35 ± 0.01 |
| IgG4 kappa | 72.9 | 0.0907 ± 0.0044 | 1.37 ± 0.23 |
| IgG4 lambda | 73.7 | 0.125 ± 0.004 | 1.10 ± 0.20 |
| CDG1 | 72.8 | 0.0803 ± 0.0010 | 1.29 ± 0.24 |
| Mab41 | 69.5 | 0.0881 ± 0.0022 | 1.93 ± 0.07 |
| Mab48 | 68.7 | 0.0785 ± 0.0035 | 2.14 ± 0.24 |

Table 4 shows the molar fraction of —SH in antibodies under non-denaturing conditions (Dulbecco's phosphate-buffered saline, pH 7.3) and denaturing conditions (Dulbecco's phosphate-buffered saline, pH 7.3 containing 5 M Gdn-HCl) calculated using a calibration curve generated using BSA as a standard. The reaction mixtures for this set of samples were incubated for 2 h at 37° C. Table 4 also shows the meting temperatures (Tm) of the first temperature dependent structural transition of these antibodies as determined by DSC.

TABLE 4

| | | Free SH | Free SH |
|---|---|---|---|
| | Tm of first | (mol —SH/mol | (mol —SH/mol |
| | thermal | of antibody) | of antibody) |
| | transition | Non-denaturing | Denaturing |
| Antibody | (° C.) | conditions | conditions |
| IgG1 kappa | 70.5 | 0.060 ± 0.003 | 0.85 ± 0.13 |
| IgG1 lambda | 60.5 | 0.204 ± 0.002 | 2.54 ± 0.09 |
| infliximab | 70 | 0.027 ± 0.001 | 0.65 ± 0.02 |
| Mab IgG1k | 66.5 | 0.114 ± 0.016 | 0.81 ± 0.16 |
| Adalimumab | 72 | 0.029 ± 0.007 | 1.14 ± 0.28 |

### Example 4

### High Through-Put Determination of Sulfhydryl to Screen and Select Library Protein Candidates

The ability to screen large numbers of samples is becoming more and more important. In order to save time, an assay that allows simultaneous analysis of a large number of samples would be advantageous.

The assay as described above (in reference to preparation of calibration curves for free sulfhydryl content) may be performed using a fluorescence compatible 96-well microtitre plate (Diagram 1). In one row of wells (e.g., A1-H1) solutions of different concentration of the —SH standard are placed for the preparation of a calibration curve. A separate calibration curve is generated for each buffer condition used in the assay. Therefore, stock solutions of the standard (BSA) are prepared using the buffer in which the antibody (protein) is analyzed. The buffers are degassed by sonication under vacuum. NPM is prepared at 10 mM concentration in dimethylformamide (DMF). For the reactions, 50 uL of the appropriate BSA solution is mixed with 250 uL of the appropriate buffer to obtain the desired final concentration. Three uL of 10 μM NPM is added to this mixture and the samples are incubated for 2 h at 25° C. (RT) for solutions in native or nondenaturing conditions, and 2 h RT or 37° C. for those with Gdn-HCl. After incubation, the reactions are stopped with 5 uL of 50% acetic acid. The fluorescence emission spectrum of the mixture is then obtained using a Fluoromax-3 fluorometer or similar fluorometer capable of scanning microtitre plates. The samples are excited at 330 nm and the fluorescence signal at 376 nm is collected. The calibration curves are prepared by plotting the fluorescence signal at 376 nm versus micromolar concentration of BSA. For analysis of free sulfhydryl, antibodies (protein) are prepared at 1 μg/mL in the desired buffer. For nondenaturing experiments, 50 uL of the Ab solution is added to 250 uL of appropriate buffer then is processed as described for the BSA standards. The fluorescence emission is determined similarly, by excitation at 330 nm while collecting the fluorescence signal at 376 nm. The micromolar concentration of —SH in the proteins is determined using the calibration curves obtained for BSA. The fraction of —SH in the proteins is determined on the basis of the final concentration of the sample in the reaction mixture. This assay can either be performed manually using a multichannel pipette, or using an automated liquid handler, such as a TECAN. The samples in microtitre plates are heated to 37° C. in a microtitre plate compatible incubator.

The results of the assay of proteins or peptides will be used to ranked stability on the basis of the —SH content.

Diagram 1. Detection of free sulfhydryl using
a microtitre plate: samples positioning.

| | 1 | 2 | 3 | 4 | 5 | 6 | 7 | 8 | 9 | 10 | 11 | 12 |
|---|---|---|---|---|---|---|---|---|---|---|---|---|
| A | B1 | S1 | B2 | S1 | B3 | S1 | B4 | S1 | B5 | S1 | B6 | S1 |
| | | B1 | | B2 | | B3 | | B4 | | B5 | | B6 |
| B | St1 | S2 | St1 | S2 | St1 | S2 | St1 | S2 | St1 | S2 | St1 | S2 |
| | B1 | B1 | B2 | B2 | B3 | B3 | B4 | B4 | B5 | B5 | B6 | B6 |
| C | St2 | S3 | St2 | S3 | St2 | S3 | St2 | S3 | St2 | S3 | St2 | S3 |
| | B1 | B1 | B2 | B2 | B3 | B3 | B4 | B4 | B5 | B5 | B6 | B6 |
| D | St3 | S4 | St3 | S4 | St3 | S4 | St3 | S4 | St3 | S4 | St3 | S4 |
| | B1 | B1 | B2 | B2 | B3 | B3 | B4 | B4 | B5 | B5 | B6 | B6 |
| E | St4 | S5 | St4 | S5 | St4 | S5 | St4 | S5 | St4 | S5 | St4 | S5 |
| | B1 | B1 | B2 | B2 | B3 | B3 | B4 | B4 | B5 | B5 | B6 | B6 |
| F | St5 | S6 | St5 | S6 | St5 | S6 | St5 | S6 | St5 | S6 | St5 | S6 |
| | B1 | B1 | B2 | B2 | B3 | B3 | B4 | B4 | B5 | B5 | B6 | B6 |
| G | St6 | S7 | St6 | S7 | St6 | S7 | St6 | S7 | St6 | S7 | St6 | S7 |
| | B1 | B1 | B1 | B1 | B3 | B3 | B4 | B4 | B5 | B5 | B6 | B6 |
| H | St7 | S8 | St7 | S8 | St7 | S8 | St7 | S8 | St7 | S8 | St7 | S8 |
| | B1 | B1 | B1 | B1 | B3 | B3 | B4 | B4 | B5 | B5 | B6 | B6 |

Rows are labeled A through H. Columns are labeled 1 through 12. B1, B2, B3, B4, B5 and B6 indicate different buffers, S indicates sample and St indicates standard sample (for the calibration curve).

### Example 5

### Method of Analyzing Free Sulfhydryl in Non-Purified Protein Samples

The ability to perform the stability testing using the free —SH method on unpurified samples, such as cell supernatants, would be advantageous. The ability to measure —SH and stability prior to column chromatography would also be advantageous. Reagents, such as NPM, are very specific to —SH; therefore, it is possible to analyze crude samples under certain conditions. Supernatants from protein expressing cell cultures grown in protein additive-free media represent a format for the use of the method of the invention.

Method for detection of free sulfhydryl in conditioned media: The samples are analyzed, ranked and selected using the manual or the automated simultaneous method described above.

What is to be claimed:

1. A method of determining the stability of a functional protein comprising the steps of
obtaining a sample of the protein,
contacting the protein sample with a chemical denaturant,
heating the protein sample in the presence of the chemical denaturant,
contacting the protein sample with a sulfhydryl reactive detection agent, and
measuring the magnitude of the signal produced by a reaction of the detection agent with sulfhydryls in the protein sample, wherein the magnitude of the signal is indicative of a lack of stability of the protein in an aqueous physiologically compatible solution.

2. The method of claim 1 which optionally includes the step of
comparing the magnitude of the signal produced by the heated, denatured sample with a signal produced by a similar sample not subjected to a chemical denaturant.

3. The method of claim 1 or 2, wherein the magnitude of the signal is compared to a calibration curve prepared using BSA under the same conditions as the protein sample.

4. The method of claim 1 or 2, wherein the detection agent exhibits enhanced fluorescence upon reaction with a free sulfhydryl.

5. The method of claim 3, wherein the detection agent is selected from the group consisting of a bimane and a maleimide derivative.

6. The method of claim 4, wherein the detection agent is N-prenyl maleimide and the fluorescence emission is read at 330ex/376em.

7. The method according to claim 1 or 2, wherein the chemical denaturant is selected from the group consisting of a guanidinium salt, acetone, urea, DMF, benzene, ammonium sulfate, a non-ionic detergent, a ionic detergents, a hydrochloric acid (HCl), acetic acid (CH3COOH), and a halogenated acetic acid.

8. The method according to claim 5, wherein the chemical denaturant is guanidinium hydrochloride or guanidinium thiocyanate.

9. The method according to claim 1 or 2, wherein the method is used to assess the relative stability of at least two purified antibody preparations.

10. The method according to claim 1, wherein the functional protein is an antibody.

11. The method according to claim 10, wherein the method is used to compare the stability of at least two antibodies.

12. Any invention described herein.
